(19) United States
(12) Patent Application Publication
(10) Pub. No.: US 2015/0182429 A1
(43) Pub. Date: Jul. 2, 2015

(54) ANTIPERSPIRANTS WITH LIMITED COATING AGENTS AND METHODS FOR PRODUCING THE SAME

(71) Applicant: The Dial Corporation, Scottsdale, AZ (US)

(72) Inventors: Debra K. Butterworth, Scottsdale, AZ (US); Catherine Schmit, Glendale, AZ (US); Jennifer Harris, Phoenix, AZ (US); Robert Demson, Cave Creek, AZ (US)

(21) Appl. No.: 14/144,741

(22) Filed: Dec. 31, 2013

(51) Int. Cl. A61K 8/28; B65B 29/10; A61Q 15/00; A61K 8/26; A61K 8/41

(52) U.S. Cl. CPC A61K 8/28; A61K 8/26; A61K 8/416; A61Q 15/00; B65B 29/10

(57) ABSTRACT

A mixed powder antiperspirant and methods for producing and using such mixed powder antiperspirants are provided. The method includes mixing an active ingredient, a film former, and a diluent to form the mixed powder antiperspirant. The use of a coating agent is limited to no more than about 10 weight percent based on the total weight of the mixed powder antiperspirant, where a coating agent is (i) any non-water soluble wax, oil, fat, or fatty alcohol with a straight chain alkane or alkene group of 10 or more carbon atoms, (ii) polyethylene, or (iii) cyclomethicone.

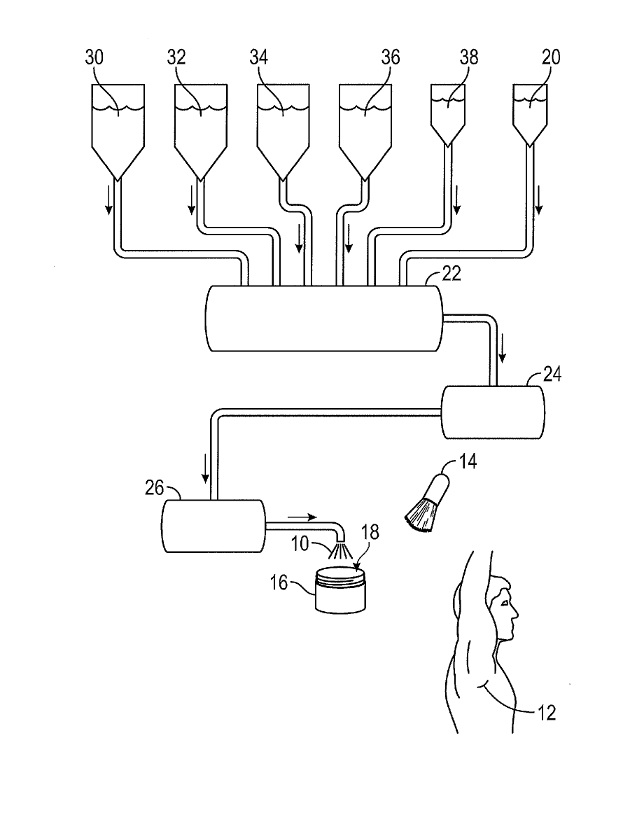

### TECHNICAL FIELD

The technical field generally relates to antiperspirants and methods for producing antiperspirants, and more particularly relates to antiperspirants with limited coating agents and methods for producing such antiperspirants.

### BACKGROUND

Antiperspirants are commonly used to control perspiration and odor caused by perspiration. Antiperspirants are available in several forms, including solid sticks, aerosols, roll-ons, gels, etc. Antiperspirants typically include an active ingredient that dissolves in the perspiration and flows with the perspiration into the top of the eccrine glands and/or the apocrine glands, otherwise known as sweat glands. The active ingredient forms a small, temporary plug near the top of the sweat gland that stops or reduces more perspiration from exiting the sweat gland. Antiperspirants are generally applied to control perspiration in the axilla or armpit, because this area typically produces large quantities of perspiration and odor compared to other parts of the body.

The various formulations of antiperspirants, such as solid sticks, roll ons, gels, and aerosols, include waxes, oils, and/or fats that aid in the application and act as diluents and carriers of the active ingredient. The hydrophobic waxes, oils, or fats are non-water soluble, and the active ingredient is water soluble, so optional rheological additives may be used to help suspend the water soluble active ingredients in the carrier medium. However, the waxes, oils, or fats tend to form a non-water soluble cover around the active ingredient, so the active ingredient is encased in non-water soluble materials. The coating decreases the efficacy of the active ingredient, because it forms a barrier that hinders the active ingredient from being dissolved in the perspiration.

Accordingly, it is desirable to provide antiperspirants and methods for producing antiperspirants with limited waxes, oils, and fats, collectively referred to as coating agents. A low concentration of coating agents can increase the efficacy of the antiperspirant. In addition, it is desirable to provide antiperspirants and methods for producing antiperspirants with limited coating agents, where the antiperspirant is a solid powder. Furthermore, other desirable features and characteristics of the present embodiment will become apparent from the subsequent detailed description and the appended claims, taken in conjunction with the accompanying drawings and this background of the invention.

### BRIEF SUMMARY

Antiperspirants with limited coating agents and methods for producing the same are provided. In an embodiment, a method is provided for producing a mixed powder antiperspirant. An active ingredient, a film former, and a diluent are mixed to form the mixed powder antiperspirant. The use of a coating agent is limited to no more than about 10 weight percent based on the total weight of the mixed powder antiperspirant, where a coating agent is (i) any non-water soluble wax, oil, fat, or fatty alcohol with a straight chain alkane or alkene group of 10 or more carbon atoms, (ii) a polyethylene, or (iii) a cyclomethicone.

In another embodiment, a method is provided for producing a mixed powder antiperspirant. An active ingredient, a film former, and a diluent are mixed to form the mixed powder antiperspirant, where the mixed powder antiperspirant includes from about 8 to about 30 weight percent (USP) active ingredient, about 0.1 to about 15 weight percent film former, and about 35 to about 95 weight percent diluent, all weights based on the total weight of the mixed powder antiperspirant. Encapsulation of the active ingredient is minimized by providing the active ingredient in a non-encapsulated form and by limiting non-water soluble compounds that encapsulate the active ingredient to about 10 weight percent or less based on the total weight of the mixed powder antiperspirant.

A mixed powder antiperspirant is provided in yet another embodiment. The mixed powder antiperspirant includes an active ingredient, a film former, and a diluent. The mixed powder antiperspirant includes no more than about 10 weight percent of a coating agent, where the coating agent is (i) any non-water soluble wax, oil, fat, or fatty alcohol with a straight chain alkane or alkene group of 10 or more carbon atoms, (ii) a polyethylene, or (iii) a cyclomethicone. All weight percents are based on the total weight of the mixed powder antiperspirant.

### BRIEF DESCRIPTION OF THE DRAWINGS

The present embodiments will hereinafter be described in conjunction with the following drawing figure, wherein:

The FIGURE is a schematic representation of a mixed powder antiperspirant and a method of producing the mixed powder antiperspirant.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

In accordance with an exemplary embodiment, a mixed powder antiperspirant is prepared as a solid in a powder form. The antiperspirant may be applied to the axilla with a powder applicator, such as a puff or brush. The mixed powder antiperspirant includes an active ingredient, a film former, and a diluent. The diluent facilitates the distribution of the antiperspirant and, hence, the active ingredient across the skin on application. The film former facilitates the retention of the active ingredient on skin. The mixed powder antiperspirant has a low concentration of hydrophobic coating agents, where coating agents are (i) non-water soluble waxes, oils, fats, or fatty alcohols, (ii) polyethylene, (iii) cyclomethicone, (iv) non-water soluble emollients, or other non-water soluble components that encapsulate the active ingredient. The substantial lack of coating agents allow perspiration to more readily dissolve the active ingredient, so a significant portion of the active ingredient in the antiperspirant can be readily dissolved, which enhances the efficacy of the antiperspirant.

Reference is made to the FIGURE. In an exemplary embodiment, the mixed powder antiperspirant 10 is a solid in powder form. As a powder, the water concentration in the mixed powder antiperspirant 10 is sufficiently low that the powder does not significantly clump or form agglomerates greater than 500 microns, or a paste. The water content that will cause clumping in the mixed powder antiperspirant 10 varies for different formulations, so the maximum allowable water content also varies. In an exemplary embodiment, the water content is about 10 weight percent (wt. %) or less based on the total weight of the mixed powder antiperspirant 10, and in another embodiment the water content is about 5 wt. % or less based on the total weight of the mixed powder antiperspirant 10.

Other liquid solvents can also cause clumping, agglomeration, or a paste form of the mixed powder antiperspirant 10, and the concentration of any other liquid solvents is sufficiently low that the powder does not have significant clumps, where clumps are aggregations of about 500 microns or more. In an exemplary embodiment, the mixed powder antiperspirant 10 has a concentration, based on the total weight of the mixed powder antiperspirant 10, of about 5 wt. % or less of any volatile organic compound, where a “volatile organic compound” is defined as a liquid organic material with a boiling point of about 30 degrees centigrade (° C.) or less at one atmosphere of pressure. In another exemplary embodiment, the mixed powder antiperspirant 10 has a volatile organic compound concentration of about 5 weight percent or less. The concentration of any other liquids is also limited such that the mixed powder antiperspirant 10 is a solid powder without significant clumps, and is not a solution, a suspension, or another liquid form.

In an exemplary embodiment, the mixed powder antiperspirant 10 is applied to the axilla 12, or other desired areas of the human body, using a powder applicator 14. The mixed powder antiperspirant 10 may be placed in a container 16 as a packed powder, but in other embodiments it is packaged as a free flowing powder. The powder applicator 14 may be packaged with the container 16 in some embodiments. The powder applicator 14 can be a brush, a puff, a cotton ball, a duster, or a wide variety of devices capable of removing the powder from the container 16 and depositing and applying it on the body at the desired location, such as the axilla 12. The powder applicator 14 and the container 16 can be designed to work together, so the powder applicator 14 is slightly smaller than an opening 18 of the container 16. The powder applicator 14 can also be designed to pick up an appropriate amount of mixed powder antiperspirant 10, where different types of applicators are more appropriate for different types of powder. For example, a brush may be more appropriate for a packed powder, and a puff may be more appropriate for a free flowing powder. In some embodiments, the mixed powder antiperspirant 10 is applied to the body directly from the container 16, without the use of a powder applicator 14, such as through a perforated cap on the container 16.

The mixed powder antiperspirant 10 is formed by combining various components in selected concentrations. The components can be combined in a wide variety of manners, as understood by those skilled in the art. In an exemplary embodiment, the components are added to a blender 22 for blending, and then optionally dried in a dryer 24, but other methods of combining the components are possible. The blender 22 can be any suitable device for blending the components, including but not limited to a vee-blender, a double cone blender, a ribbon blender, a plow mixer, and the like. Alternatively, the components can be dissolved, slurried, or otherwise incorporated into a liquid, and then mixed in a liquid or semi-liquid state. The liquid component can then be evaporated to provide the mixed powder. The dryer 24 is used if the removal of liquids is desired. The liquids that can be removed include water, volatile organic compounds, or other liquids as discussed above. Many suitable dryers 24 can be used, including but not limited to drum dryers, rotary dryers, spray dryers, etc. In some embodiments, the particle size of the mixed powder antiperspirant 10 is reduced in a size reduction device 26. A wide variety of size reduction devices 26 can be used in various embodiments, including but not limited to hammer mills, impact mills, ball mills, etc. The size reduction device 26 may also include screens to separate large particles from the mixed powder antiperspirant 10. In various embodiments, the blender 22, dryer 24, and size reduction device 26 can be used in different orders. Each of the blender 22, dryer 24, and size reduction device 26 are optional in various embodiments.

As noted above, the various embodiments of the mixed powder antiperspirant 10 comprise a water-soluble active antiperspirant compound. Active antiperspirant compounds contain at least one active ingredient 30, typically metal salts, that mayo reduce perspiration by diffusing through the sweat ducts and polymerizing in the sweat ducts, where they combine with proteins to form an amorphous metal hydroxide agglomerate, plugging the sweat ducts so perspiration cannot diffuse to the skin surface. Some active ingredients 30 that may be used in the mixed powder antiperspirant 10 include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zirconyl hydroxy chloride conforming to the above-described formulas. Examples of active ingredients 30 suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the active ingredient 30 is aluminum zirconium pentachlorohydrex glycine complex or aluminum zirconium trichlorohydrex glycine complex. In a more preferred embodiment, the antiperspirant product comprises an active ingredient 30 at an active level of about 8 to about 30 wt. % (USP) of the total mixed powder antiperspirant 10. As used herein, weight percent (USP) or wt. % (USP) of an antiperspirant salt is calculated as anhydrous weight percent in accordance with the U.S.P. method, as is well known in the art. This calculation excludes any bound water and glycerin. In a most preferred embodiment, the antiperspirant product comprises about 15-25 wt. % (USP) aluminum zirconium pentachlorohydrex glycine complex or aluminum zirconium trichlorohydrex glycine complex.

In many embodiments, the active ingredient 30 is non-encapsulated or substantially non-encapsulated, so it is freely soluble upon application. “Non-encapsulated” means there are no polymeric coatings, wax coatings, fat coatings, or other hydrophobic coatings surrounding the active ingredient 30 and hindering dissolution in aqueous mediums. In some embodiments, most of the active ingredient 30 is non-encapsulated, such as about 90 weight percent or more of the active ingredient 30 is non-encapsulated, based on the total weight of the active ingredient 30.

In some embodiments, the mixed powder antiperspirant 10 contains film formers 32. Film formers 32 are compounds that facilitate the formation of a pliable, cohesive, and continuous covering of the mixed powder antiperspirant 10 over the skin. In some embodiments, the film former 32 is water soluble, where a compound is defined as “water soluble” if it forms about a 0.1 wt. % solution or more in water at 25° C. Compounds that are not water soluble are considered non-water soluble. The film former 32 is also hydrophilic in some embodiments, so water or perspiration is attracted to the film former 32. In many embodiments, the film former 32 is a polymer. Skin has a net negative charge, so some film formers 32 include a salt where a negative ion dissociates from the polymer salt in aqueous mediums, such as perspiration. Once the negative ion dissociates, the film former 32 has a net positive charge that more readily adheres to the skin. Examples of materials suitable for use as film formers 32 in the mixed powder antiperspirant 10 include, but are not limited to, chitosan, modified cornstarch, cellulose polymers, polyacrylate polymers, polyvinyl pyrrolidones, synthetic wax polymers, and combinations thereof. The film former 32 may be included in the mixed powder antiperspirant 10 at concentrations ranging from about 0.1 to about 15 wt. % in some embodiments, from about 0.1 to about 5 wt. % in other embodiments, and from about 0.1 to about 1 wt. % in yet other embodiments, based on the total weight of the mixed powder antiperspirant 10.

In another exemplary embodiment, the mixed powder antiperspirant further includes a diluent 34 that serves as a carrier in the mixed powder antiperspirant 10. The diluent 34 is water soluble in some embodiments, but non-water soluble diluents 34 can also be used. Many compounds can be used as the diluent 34, including talc, corn starch, or starches from other sources such as rice, tapioca, soy, turnip, manioc, sorghum potato, or many other plants. Other suitable diluents 34 include titanium oxide, zinc oxide, kaolin, colloidal silica, chalk, calcium phosphate, magnesium carbonate, zinc stearate, magnesium stearate, and others. The diluent 34 is added in sufficient quantities to form about 35 wt. % (US) to about 95 wt. %, based on the total weight of the mixed powder antiperspirant 10 in some embodiments. In other embodiments, the diluent 34 is added to form about 50 to about 80 wt. % based on the total weight of the mixed powder antiperspirant 10, and in yet other embodiments the diluent 34 forms about 60 to about 75 wt. % based on the total weight of the mixed powder antiperspirant 10.

The mixed powder antiperspirant 10 also may contain an additive 36 typically used in antiperspirants. The additive 36 may be one or more of a perfume, dye, flow agent, bactericide, odor control agent, antioxidant, preservative, moisture absorber, or other compound that imparts desired properties or characteristics to the mixed powder antiperspirant 10. The additive 36 may include one or more of many different categories of additives 36, and may be included in the mixed powder antiperspirant 10 at a concentration from 0 to about 25 wt. % based on the total weight of the mixed powder antiperspirant 10. In other embodiments, the additive 36 is present from about 0.1 to about 25 wt. %, such as from about 0.1 to about 5 wt. % based on the total weight of the mixed powder antiperspirant 10.

As noted above, the ingredients can be mixed and combined in powder form. Therefore, in this embodiment, there is little or no need for a rheological additive 20 to suspend an active ingredient(s) 30. Various rheological additives 20 are available for antiperspirants, such as silica (including fumed silica), other silicon compounds, hectorite clays, organoclays including hectorite clay as a component, or other rheological additives 20. In an exemplary embodiment, the concentration of any rheological additives 20 is about 3 wt. % or less, based on the total weight of the mixed powder antiperspirant 10, and in another embodiment about 1 wt. % or less.

As noted above, the active ingredient 30 is non-encapsulated or substantially non-encapsulated because a limited amount of coating agent 38 or no coating agent 38 is present in the mixed powder antiperspirant 10 and because no polymers or coating agents 38, or substantially no polymers or coating agents are fowled around the active ingredient prior to blending in the mixed powder antiperspirant 10. A coating agent 38 includes (i) any non-water soluble wax, oil, fat, or fatty alcohol comprising a straight chain alkane or alkene group of 10 or more carbon atoms, (ii) polyethylene, and (iii) cyclomethicone. The straight chain alkane or alkene group tends to form a coating that encapsulates the active ingredient 30. The term “fat” is defined to include non-water soluble esters of triglycerides, as well as non-water soluble fatty acids or esters. Some of the compounds added to the mixed powder antiperspirant 10 may be a coating agent 38, but the concentration of any such material is limited such that most of the active ingredient 30 is non-encapsulated. For example, if a synthetic wax polymer film former 32 is used, the synthetic wax polymer film former 32 is present in a low concentration to minimize encapsulation of the active ingredient 30, such as a concentration of about 5 wt. % or less in some embodiments, or 1 wt. % or less in other embodiments, based on the total weight of the mixed powder antiperspirant 10. In an exemplary embodiment, the coating agent 38 is limited to a total of about 10 wt.% or less based on the total weight of the mixed powder antiperspirant 10, but in other embodiments the coating agent 38 is limited to about 5 wt. % or less, for example, about 2 wt. % or less, such as about 1 wt. % or less based on the total weight of the mixed powder antiperspirant 10. The limited quantity of a coating agent 38 provides a mixed powder antiperspirant 10 with a non-encapsulated active ingredient 30 readily available for use.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

What is claimed is:

1. A method of producing a mixed powder antiperspirant, the method comprising the steps of:
combining an active ingredient, a film former, and a diluent to form the mixed powder antiperspirant; and
limiting a coating agent to no more than about 10 weight percent based on the total weight of the mixed powder antiperspirant, wherein the coating agent is (i) any non-water soluble wax, oil, fat, or fatty alcohol comprising a straight chain alkane or alkene group of 10 or more carbon atoms, (ii) a polyethylene, or (iii) a cyclomethicone.

2. The method of claim 1 further comprising:
drying the mixed powder antiperspirant such that a water content is less than about 5 weight percent, and a volatile organic compound content is less than about 5 weight percent, the weights based on the total weight of the mixed powder antiperspirant, wherein a volatile organic compound is an organic compound with a boiling point of about 30° C. or less.

3. The method of claim 1 wherein limiting the coating agent further comprises limiting the coating agent to no more than about 2 weight percent of the mixed powder antiperspirant based on the total weight of the mixed powder antiperspirant.

4. The method of claim 1 wherein limiting the coating agent further comprises limiting the coating agent to no more than about 1 weight percent based on the total weight of the mixed powder antiperspirant.

5. The method of claim 1 further comprising:
combining an additive with the mixed powder antiperspirant, wherein the additive comprises from about 0.1 to 25 weight percent based on the total weight of the mixed powder antiperspirant.

6. The method of claim 1 further comprising:
placing the mixed powder antiperspirant in a container; and
packaging the container with a powder applicator.

7. The method of claim 1 wherein combining the active ingredient, the film former, and the diluent to form the mixed powder antiperspirant further comprises combining the active ingredient, the film framer, and the diluent wherein the active ingredient is chosen from aluminum halides, aluminum chlorohydrates, aluminum hydroxyhalides, zirconyl oxyhalides, zirconyl hydroxyhalides, or combinations thereof.

8. The method of claim 1 wherein combining the active ingredient, the film former, and the diluent to form the mixed powder antiperspirant further comprises combining the active ingredient, the film former, and the diluent wherein the film former is chosen from chitosan, modified cornstarch, cellulose polymers, polyacrylate polymers, polyvinyl pyrrolidones, synthetic wax polymers, and combinations thereof.

9. The method of claim 1 further comprising:
forming the mixed powder antiperspirant into a packed powder in a container.

10. The method of claim 1 wherein combining the active ingredient, the film former, and the diluent to form the mixed powder antiperspirant further comprises combining the active ingredient, the film former, and the diluent to form the mixed powder antiperspirant wherein 90 weight percent or more of the active ingredient is non-encapsulated, based on the total weight of the active ingredient.

11. The method of claim 1 wherein combining the active ingredient, the film former, and the diluent to form the mixed powder antiperspirant further comprises combining the active ingredient, the film former, and the diluent wherein the film former is hydrophilic, and wherein the film former comprises a salt.

12. The method of claim 11 wherein combining the active ingredient, the film former, and the diluent to form the mixed powder antiperspirant further comprises combining the active ingredient, the film former, and the diluent wherein the film former is water soluble.

13. The method of claim 1 further comprising:
limiting a rheological additive to about 1 weight percent or less based on the total weight of the mixed powder antiperspirant.

14. A method of producing a mixed powder antiperspirant, the method comprising the steps of:
combining an active ingredient, a film former, and a diluent to form the mixed powder antiperspirant, wherein the active ingredient comprises from about 8 to about 30 weight percent (USP), the film former comprises from about 0.1 to about 15 weight percent, and the diluent comprises from about 35 to about 95 weight percent, all weights based on the total weight of the mixed powder antiperspirant; and
minimizing encapsulation of the active ingredient by providing the active ingredient in a non-encapsulated form and by limiting non-water soluble compounds that encapsulate the active ingredient to about 10 weight percent or less based on the total weight of the mixed powder antiperspirant.

15. A mixed powder antiperspirant comprising:
an active ingredient;
a film former; and
a diluent;
wherein the mixed powder antiperspirant comprises no more than 10 weight percent of a coating agent, wherein the coating agent is (i) any non-water soluble wax, oil, fat, or fatty alcohol with a straight chain alkane or alkene group of 10 or more carbon atoms, (ii) a polyethylene, or (iii) a cyclomethicone, and where all weight percents are based on the total weight of the mixed powder antiperspirant.

16. The mixed powder antiperspirant of claim 15 wherein the mixed powder antiperspirant is a free flowing powder.

17. The mixed powder antiperspirant of claim 15 wherein the mixed powder antiperspirant comprises no more than about 5 weight percent of the coating agent based on the total weight of the mixed powder antiperspirant.

18. The mixed powder antiperspirant of claim 15 wherein the mixed powder antiperspirant comprises no more than about 1 weight percent of the coating agent based on the total weight of the mixed powder antiperspirant.

19. The mixed powder antiperspirant of claim 15 further comprising an additive present in an amount of from about 0.1 to about 5 weight percent based on the total weight of the mixed powder antiperspirant.

20. The mixed powder antiperspirant of claim 15 wherein the mixed powder antiperspirant comprises about 5 weight percent or less of water, and wherein the mixed powder antiperspirant comprises about 5 weight percent or less of any volatile organic compound with a boiling point of about 30° C. or less, based on the total weight of the mixed powder antiperspirant.
